SEQUENCE LISTING (10 sequences)

SEQ ID NO: 1 - RNA, 20 nt, Burkholderia pseudomallei
  source 1..20: mol_type = other RNA
tcgtaacataattgccgggc

SEQ ID NO: 2 - RNA, 20 nt, Burkholderia pseudomallei
  source 1..20: mol_type = other RNA
gacgatcgcctccgttcttg

SEQ ID NO: 3 - RNA, 20 nt, Burkholderia pseudomallei
  source 1..20: mol_type = other RNA
actacatacccactattcag

SEQ ID NO: 4 - RNA, 20 nt, Burkholderia pseudomallei
  source 1..20: mol_type = other RNA
ccgccacccaatccgagtca

SEQ ID NO: 5 - RNA, 20 nt, Burkholderia pseudomallei
  source 1..20: mol_type = other RNA
agttcgaggcggtccgaagc

SEQ ID NO: 6 - RNA, 20 nt, Burkholderia pseudomallei
  source 1..20: mol_type = other RNA
gtgcgcgaagttatgtggca

SEQ ID NO: 7 - RNA, 20 nt, Burkholderia pseudomallei
  source 1..20: mol_type = other RNA
atctcaagacgacgtccaat

SEQ ID NO: 8 - RNA, 20 nt, Burkholderia pseudomallei
  source 1..20: mol_type = other RNA
aatggtcgcgaacccaccgt

SEQ ID NO: 9 - RNA, 21 nt, unidentified
  source 1..21: mol_type = other RNA; note = Lachnospiraceae bacterium ND2006
taatttctactaagtgtagat

SEQ ID NO: 10 - RNA, 19 nt, Acidaminococcus sp.
  source 1..19: mol_type = other RNA
aatttctactcttgtagat

(19) United States
(12) Patent Application Publication
(10) Pub. No.: US 2026/0117313 A1
(43) Pub. Date: Apr. 30, 2026

(54) A CRISPR RNA FOR DETECTION OF BURKHOLDERIA PSEUDOMALLEI-ASSOCIATED GENETIC MATERIAL IN A BIOLOGICAL SAMPLE AND A METHOD USING THE SAME

(71) Applicant: Chiang Mai University, Chiang Mai (TH)

(72) Inventors: Somsakul WONGPALEE, Chiang Rai (TH); Kamolchanok CHEWAPREECHA, Bangkok (TH); Hathairat THANANCHAI, Chiang Mai (TH); Henrik B. ROSLUND, San Jose, CA (US)

(21) Appl. No.: 18/993,824

(22) PCT Filed: Jul. 26, 2023
(86) PCT No.: PCT/TH2023/050018

§ 371 Date: Jan. 13, 2025

(30) Foreign Application Priority Data
Jul. 26, 2022 (TH) 2201004704

(51) Int. Cl. C12Q 1/689; C12N 9/22; C12N 15/11

(52) U.S. Cl. CPC C12Q 1/689; C12N 9/226; C12N 15/11; C12N 2310/20

(57) ABSTRACT

Provided is a CRISPR RNA (crRNA) applicable in a CRISPR-based ribonucleoprotein (RNP) system for reacting towards one or more genetic materials derived from Burkholderia pseudomallei in a biological sample. The crRNA comprises a spacer region having one of a poly nucleotide sequence selected from SEQ ID No. 3. SEQ ID No. 5, and SEQ ID No. 7: a repeat region preceding the spacer region forming a secondary structure thereof; and a first extension region arranged immediately after the spacer region.

### TECHNICAL FIELD

The present disclosure relates to a modified CRISPR RNA (crRNA) applicable in a CRISPR-based ribonucleoprotein (RNP) system for detecting presence of genomic DNA of Burkholderia pseudomallei, a causative agent of Melioidosis, in a subject. More specifically, the disclosed crRNA can be employed in a lateral flow assay for detecting presence of Burkholderia pseudomallei in a biological sample.

### BACKGROUND

Burkholderia pseudomallei is an environmental Gram-negative bacillus found mostly in soil and water, and a causative agent of melioidosis a rapidly fatal infectious disease endemic in many tropical and subtropical countries [1-2]. Septicemic melioidosis can lead to death in less than 48 hours if not diagnosed and treated properly [3]. Human hosts can acquire B. pseudomallei by skin inoculation, ingestion of contaminated food or water supply, or inhalation of aerosolized bacterium. Infected individuals can display a variety of clinical manifestations ranging from pneumonia, organ abscesses to septicemia [4], which make clinical diagnosis challenging. The mortality rate of infected patients is high, reported as 35-40% in Thailand, despite availability of antibiotic treatments [1,5]. This is, in part, due to delayed- or missed diagnosis, which often leads to delayed correct treatments. The gold standard laboratory diagnosis for melioidosis remains the culturing of B. pseudomallei from clinical specimens, followed by a panel of biochemical tests [6]. This diagnosis can take several days, requires an enhanced biosafety level 2 (BSL2 enhanced) laboratory and sometimes generates inconsistent results [4,7]. Since timely and optimal treatments of melioidosis affect outcomes of the disease [4,7,8], rapid and accurate identification of B. pseudomallei is much needed.

Serological diagnosis of B. pseudomallei-specific antigens or antibodies, such as indirect hemagglutination (IHA), enzyme linked immunosorbent assay (ELISA), immunochromatographic assay (ICT) and lateral flow immunoassay (LFI) have been developed [9-16]. However, interpretation of these serological tests has proven to be complicated in endemic areas where past infection or prior exposure to the bacteria causes seroconversion without active infection. In addition, heterogeneity in antibody response to B. pseudomallei was reported in patients, thereby requiring testing of multiple antigens to confirm the result. Moreover, some B. pseudomallei culture-confirmed patients were seronegative during acute infection. Together, these limit the applications of serological assays as diagnostic tools for melioidosis. In addition to serological assays, real-time polymerase chain reaction (real-time PCR)-based assays have been developed to detect the DNA of B. pseudomallei [17,18]. Although these methods showed high sensitivity and specificity, real-time PCR-based assays require expensive real-time PCR machines, which may not be readily available in many laboratories. As alternatives, isothermal DNA amplifications such as loop-mediated isothermal amplification (LAMP) and recombinase polymerase amplification (RPA) [20,21] have been developed to overcome this dependency on expensive equipment. Both assays exhibit very sensitive detection. RPA, for example, has a limit of detection (LOD) from between 25-50 copies of B. pseudomallei genomic DNA. Albeit promising, these assays are based on primers designed to target a genomic locus, such as the type 3 secretion system-1 (T3SS-1), of few selected reference strains. Their detection coverage on broader B. pseudomallei population is currently unknown. Furthermore, specificity of RPA assay remains to be further determined as it can tolerate some mismatches in primer sequences.

Cluster regularly interspaced short palindromic repeat (CRISPR) is an adaptive defence system in bacteria and archaea that provides sequence-specific immunity against invading nucleic acids such as bacteriophages or plasmids [22]. A CRISPR locus incorporates remnants of genetic material from past infections and uses them for RNA-guided endonuclease against future infections. This RNA-guided endonuclease, known as a CRISPR-Cas complex, consists of a CRISPR RNA (crRNA) and CRISPR-associated (Cas) nuclease(s). The complex recognizes a target site through crRNA-mediated base-pairing complementarity before initiating its cleavage. Various CRISPR-Cas systems have been identified, each having a unique property toward nucleic acid [23,24]. One prominent system is CRISPR-Cas9, a system that has been exploited for precise genome editing both in research fields and in gene therapy [25], and for potential therapeutics for infectious diseases such as human immunodeficiency virus (HIV) and hepatitis B virus (HBV) [26,27]. CRISPR-Cas12a and -Cas13a are among recently discovered members of the CRISPR-Cas family, which recognize DNA and RNA targets, respectively. Their initial target recognitions activate their non-specific endonucleases that collaterally cut other non-target nucleic acids in their vicinity [28,29]. This collateral cleavage property of the two CRISPR-Cas systems has been used as a biosensor to induce cleavage of fluorescent nucleic acid probes, allowing a sensitive and a quantifiable readout of the actual target nucleic acids [30]. By coupling this property of CRISPR-Cas12a or -13a with another signal enhancement step such as metal-enhanced fluorescence (MEF), autocatalysis-driven feedback amplification or an isothermal pre-amplification of nucleic acids, the CRISPR-Cas systems can be used as detection platforms that offer superior sensitivity and specificity [31-34]. Examples are detection assays called DNA endonuclease-targeted CRISPR trans reporter (DETECTR) and specific high-sensitivity enzymatic reporter unlocking (SHERLOCK), which couple isothermal pre-amplification of nucleic acids with the CRISPR-Cas12a and -Cas13a systems, respectively. In view of that, it is possible to develop a simplified and time-saving diagnostic tool or kit for melioidosis using the CRISPR-Cas-based technology to address some of the shortcomings found in the current clinical practice as discussed above.

### SUMMARY

The present disclosure aims to provide one or more engineered or modified crRNAs usable in detecting presence of Burkholderia pseudomallei-associated or -specific genetic material in a biological sample. Based upon presence or absence of the Burkholderia pseudomallei-associated genetic material in the biological sample, the present disclosure allows acquiring at least a preliminary diagnosis about melioidosis in a subject, whom the biological sample derived from.

Further object of the present disclosure is to furnish one or more crRNAs operable in a CRISPR-based RNP system for detecting Burkholderia pseudomallei-associated genetic material in a biological sample. The CRISPR-based RNP system facilitates the detection of Burkholderia pseudomallei-associated genetic material with relatively short processing time compared to other common clinical or laboratory approaches used.

Still, another object of the present disclosure is to offer a method utilizing the mentioned crRNA along with the assembled CRISPR-based RNP system in running a biological sample for identifying or detecting presence of Burkholderia pseudomallei-associated genetic materials thereto and subsequently deduce at least a preliminary diagnosis about potential infection relating to melioidosis.

At least one of the preceding objects is met, in whole or in part, by the present disclosure, in which one of the embodiments of the present disclosure is a single guide RNA (crRNA) applicable in a CRISPR-based ribonucleoprotein (RNP) system for reacting towards one or more genetic materials derived from Burkholderia pseudomallei in a biological sample. The crRNA comprises a spacer region having one of polynucleotide sequences selected SEQ ID No. 3, SEQ ID No. 5, and SEQ ID No. 7; a repeat region preceding the spacer region forming a secondary structure thereof; and a first extension region arranged immediately after the spacer region.

For more embodiments, the spacer region is being configured to hybridize on a complementary sequence resided in the genetic materials and the complementary sequence is preceded with a protospacer adjacent motif (PAM) comprising a sequence of TTTV with V being a non-T nucleotide.

For more embodiments, the crRNA comprises a second extension region having a sequence of Gs, preferably GGG being arranged preceding of the repeat region.

For more embodiments, the repeat region comprises a polynucleotide sequence of SEQ ID No. 9 or SEQ ID No. 10 or a polynucleotide sequence at least 80% sequence identity of SEQ ID No. 9.

For more embodiments, the first extension region comprises a polynucleotide sequence of AAAGGAA.

Preferably, in some embodiments, the CRISPR-based RNP system is a CRISPR-Cas12a RNP system, a CRISPR-Cas12b RNP system, or a CRISPR-Cas13 RNP system.

Another aspect of the present disclosure is associated to a method for detecting presence of one or more genetic materials of Burkholderia pseudomallei acquired from a biological sample comprising the steps of: providing the one or more genetic materials of Burkholderia pseudomallei; assembling of a CRISPR-Based RNP system, the assembled CRISPR-Based RNP system comprising a crRNA with a spacer region having one of a polynucleotide sequences selected from SEQ ID No. 3, SEQ ID No. 5 and SEQ ID No. 7; reacting the genetic materials with the assembled CRISPR-based RNP system along a plurality of reporter probes in a buffer solution, the CRISPR-Based RNP system being configured to activate the cleavage of reporter probes for emitting a fluorescent signal upon hybridizing on a polynucleotide sequence complementary to the spacer region located within the one or more genetic materials; and detecting the emitted signal to confirm presence of the B. pseudomallei in the provided biological sample.

In some embodiments of the disclosed method, the detecting step further comprises subjecting portion of the reacted buffer solution to a lateral flow assay for capturing the emitted signal. The reacted buffer may contain the end products of the reaction among the preassembled CRISPR-based RNP system and the genetic material such as partial or whole genome of the Burkholderia pseudomallei DNA. Preferably, the lateral flow assay can be a dipstick constructed to capture then visualize detectable moieties or signals released from the signaling probes.

In some embodiments of the disclosed method, the crRNA further comprises a repeat region preceding the spacer region forming a secondary structure thereof and the repeat region comprises a polynucleotide sequence of SEQ ID No. 9 or SEQ ID No. 10.

In some embodiments of the disclosed method, the crRNA further comprises a first extension region arranged immediately after the spacer region and the first extension region comprises a polynucleotide sequence of AAAGGAA. The addition of the first extension favours the collateral cleavage activities upon hybridizing the crRNA onto the target sequence positioned in the genetic materials of the biological sample. Also, in some embodiments of the disclosed method, the crRNA further comprises a second extension region having a sequence of GGG being arranged preceding of the repeat region.

Preferably, the CRISPR-based RNP system is a CRISPR-Cas12a RNP system derived from Lachnospiraceae bacterium ND2006 in several embodiments of the disclosed method.

In a number of the embodiments of the disclosed system, the spacer region is being configured to hybridize on a complementary sequence resided in the genetic materials and the complementary sequence is preceded with a protospacer adjacent motif (PAM) comprising a sequence of TTTV with V being a non-T nucleotide.

### BRIEF DESCRIPTION OF THE DRAWINGS

FIG. 1A shows a schematic representation of CRISPR-Cas12a developed in the present disclosure recognizing its target DNA;

FIG. 1B illustrates a phylogeny map of global B. pseudomallei population (n=3,341) collected between years of 1935 and 2018 with the outer ring representing major lineages denoted as the Burkholderia Bayesian cluster (bb) 1 to 22 and the points located in the inner ring highlight 30 representative genomes used for CRISPR-Cas12a target scanning;

FIG. 1C is a schematic representation of the bioinformatic analysis used for identifying B. pseudomallei-specific CRISPR-Cas12a target sites including CRISPR selection and CRISPR filtering;

FIG. 2A shows a schematic representation of DETECTR assay;

FIG. 2B is a graph showing collateral cleavage activity of crBP34;

FIG. 2C is a graph showing collateral cleavage activity of crBP36;

FIG. 2D is a graph showing collateral cleavage activity of crBP38;

FIG. 3A is a heatmap showing fluorescent signals generated by crBP34-DETECTR assay;

FIG. 3B is a heatmap showing fluorescent signals generated by real-time PCR assay;

FIG. 3C is a schematic representation of a lateral flow dipstick readout developed by the present disclosure, where RPA and CRISPR reactions were performed similar to FIG. 2A, except that FAM-Quencher probes are replaced with FAM-Biotin probes (F-B);

FIG. 3D is a picture showing lateral flow dipstick readout retains sensitivity of the crBP34-DETECTR assay; and

FIG. 4 is a graph showing the result of crBP34-DETECTR assay towards various clinical isolates including B. pseudomallei.

### DETAILED DESCRIPTION

Hereinafter, the disclosure shall be described according to the preferred embodiments and by referring to the accompanying description and drawings. However, it is to be understood that referring the description to the preferred embodiments of the disclosure and to the drawings is merely to facilitate discussion of the various disclosed embodiments and it is envisioned that those skilled in the art may devise various modifications without departing from the scope of the appended claim.

The term “polynucleotide” or “nucleic acid” as used herein designates mRNA, RNA, CRNA, cDNA or DNA, for examples. The term typically refers to oligonucleotides greater than 30 nucleotide residues in length.

The term “genetic materials’ used herein throughout the specification shall refer to whole or partial genome of Burkholderia pseudomallei extracted, duplicated and/or amplified using known approaches from the organism, which is preferably obtained from the bodily fluid of a subject or a patient. The organism obtained from the bodily fluid may be subjected to further culturing to increase the number of Burkholderia pseudomallei and copies of the genetic materials associated thereto. Also, the genetic materials may undergo one or more pretreatment steps prior to performing the disclosed method.

The term “CRISPR RNA” or “crRNA” used herein the specification refers to an RNA molecule that plays a pivotal role in the CRISPR-Cas system, acting as a guide molecule to direct Cas proteins toward specific DNA sequences for precise targeting. It typically comprises a repeat and a spacer, which facilitate interactions with the cognate Cas enzyme and the target DNA, respectively. The spacer is usually 17-24 nucleotide in length, complementary to the target DNA, which preferably a conserved nucleotide sequence retained in the genetic material of Burkholderia pseudomallei.

The terms “crBP32”, “crBP33”, “crBP34”, “crBP35”, “crBP36”, “crBP37”, “crBP38” and “crBP39” used in the detailed description particularly in the examples section shall respectively refer to SEQ ID No. 1, SEQ ID No. 2, SEQ ID No. 3, SEQ ID No. 4, SEQ ID No. 5, SEQ ID No. 6, SEQ ID No. 7 and SEQ ID No. 8 unless mentioned otherwise.

In accordance with one aspect of the present disclosure, a crRNA applicable in a CRISPR-based RNP system for reacting towards one or more genetic materials derived from Burkholderia pseudomallei in a biological sample is disclosed. The crRNA essentially comprises a spacer region having one of a polynucleotide sequence selected from SEQ ID No. 3, SEQ ID No. 5, and SEQ ID No. 7; a repeat region preceding the spacer region forming a secondary structure thereof; and a first extension region arranged immediately after the spacer region. The B. pseudomallei in the biological sample may undergo a pretreatment or preconditioning like being cultured in a suitable medium to increase the number of B. pseudomallei and the genetic material associated thereto. For some embodiments, the cultured and isolated bacteria of B. pseudomallei from the patient or the subject is the biological sample. For more embodiments, the biological sample may be a bodily fluid such as blood harvested from the subject or patient containing sufficient number or amount of the genetic materials of B. pseudomallei for a reaction with the established CRISPR-based RNP system using the disclosed crRNA. Likewise, the bodily fluid may be pretreated to concentrate the genetic materials of B. pseudomallei and/or remove any components potentially adversely affecting the CRISPR traction. It is possible also in some embodiments to have the segment of gene and/or non-coding sequence of the B. pseudomallei genetic materials complementary to the sequence of the spacer region firstly amplified through a thermal-cycling process to acquire high concentrate of amplicons carrying the complementary sequences followed by reacting the amplicons with the CRISPR-based RNP system.

It is important to note that each of the SEQ ID No. 3, SEQ ID No. 5 and SEQ ID No. 7 (respectively corresponding to crBP34, crBP36 and crBP38 in part of the description) in the crRNA is capable of hybridizing onto a complementary sequence, which is resided in conserved region of the DNA of over 90% of the known strains of B. pseudomallei worldwide as indicated in the examples given hereinafter. Employing these sequences, which are highly specific towards B. pseudomallei, as the spacer region has rendered the constructed crRNA and the CRISPR-based RNP system assembled thereto the effective tools to search and identify presence of B. pseudomallei in a biological sample thus at least deducing a preliminary diagnosis about B. pseudomallei infection.

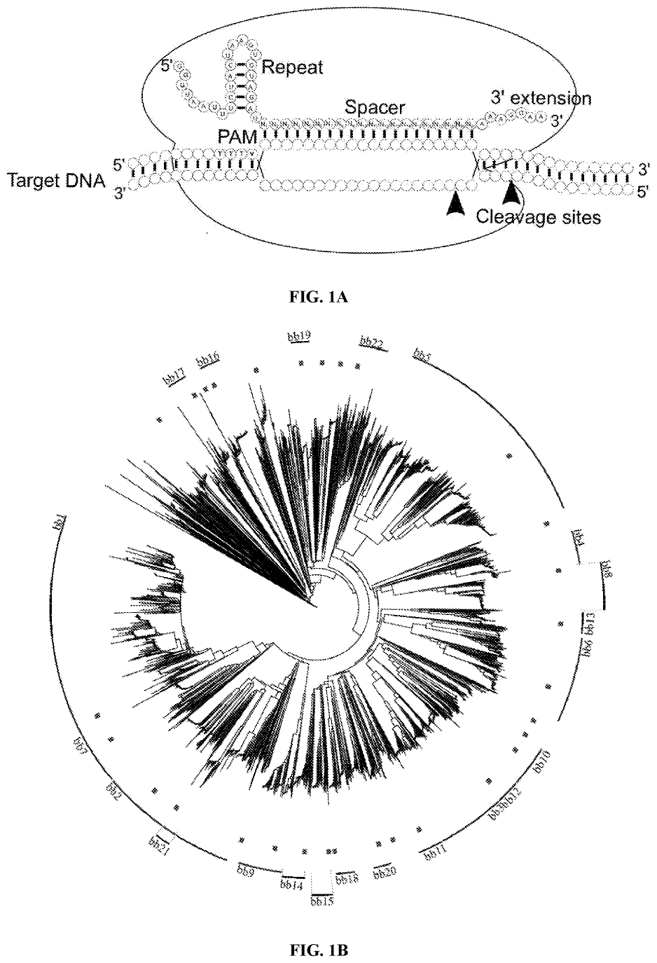

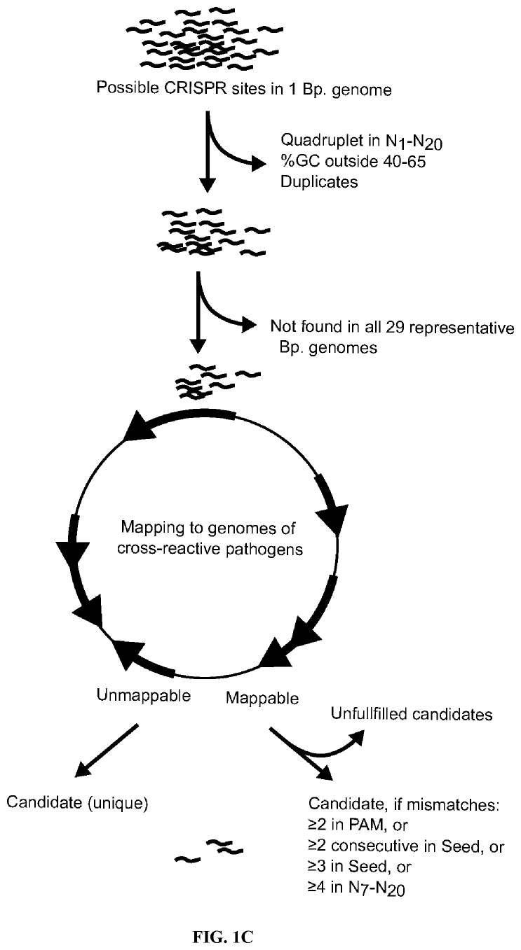

According to several preferred embodiments, the CRISPR-based RNP system is a CRISPR-Cas12a RNP system or a CRISPR-Cas12a RNP complex. The Cas12a ribonuclease is an RNA-guide endonuclease having a capacity of collateral cleavage once activated and within an environment provided with single-stranded deoxyribonucleic acid, ssDNA. As illustrated in FIG. 1A, Cas12a protein (grey lobes) needs to interact with the crRNA, which is composed of two essential elements—a repeat region and a spacer region. The repeat region is a structural scaffold of the crRNA, required for crRNA-Cas12a interaction, while the spacer is used for target recognition.

Furthermore, in some embodiments, the first extension region comprises a polynucleotide sequence of AAAGGAA. Addition of the first extension in these embodiments has shown improved collateral cleavage activities thus enhancing detectability of signals or a plurality of signalling moieties released upon recognizing presence of the B. pseudomallei genetic materials in the biological sample. As stated above, the first extension region is positioned immediately after the spacer region or at 3′-end of the space region.

Another effort of the present disclosure to improve specificity of the disclosed crRNA towards B. pseudomallei is by way of identifying a particular protospacer adjacent motif (PAM) associated to the sequence complementary to the spacer region resided in the genetic materials of B. pseudomallei. In more specific, the spacer region is being configured to hybridize on a complementary sequence resided in the genetic materials and the complementary sequence is preceded with a PAM having a sequence of TTTV with V being a non-T nucleotide. The disclosed crRNA utilizes the PAM sequence as an extra measure to verify that the complementary sequence present in the genetic materials is indeed originated from B. pseudomallei rather than other common causes of bacterial infection from common causes of bacterial infections such as S. pyogenes, S. epidermidis, K. pneumoniae, S. agalactiae, P. aeruginosa, B. thailandensis, S. pneumoniae, E. coli, S. aureus, and A. baumannii. PAM is a prerequisite to activate the CRSPR-based RNP system for initiating the trans cleavage of the complementary sequence.

To effectuate attachment of the crRNA onto the Cas endonuclease, the repeat region forming the scaffold structure preferably comprises a polynucleotide sequence of SEQ ID No. 9. The polynucleotide sequence of SEQ ID No. 9 folds naturally into a secondary structure to adaptably anchor the entire gRNA or crRNA onto the Cas endonuclease assembling or establishing the CRISPR-based RNP system. In more embodiments, the crRNA comprises a second extension region having a sequence of GGG being arranged preceding of the repeat region.

As mentioned in the setting forth, another aspect of the present disclosure relates to a method for detecting presence of one or more genetic materials of Burkholderia pseudomallei acquired from a biological sample. Any DNA extraction procedure may be performed to derive the DNA extracts, which represent the genetic materials of each pathogen. Preferably, the disclosed method comprises the steps of providing the one or more genetic materials of Burkholderia pseudomallei; assembling of a CRISPR-based RNP system, the assembled CRISPR-based RNP system comprising a crRNA with a spacer region having one of a polynucleotide sequence selected from SEQ ID No. 3, SEQ ID No. 5, and SEQ ID No. 7; reacting the genetic materials with the assembled CRISPR-based RNP system along a plurality of reporter probes in a buffer solution, the CRISPR-Based RNP system being configured to activate the reporter probe for emitting a signal upon hybridizing on a polynucleotide sequence complementary to the spacer region located within the one or more genetic materials; and detecting the emitted signal to confirm presence of the Burkholderia pseudomallei in the biological sample.

Similarly, the B. pseudomallei in the biological sample of the disclose method may undergo a pretreatment or preconditioning like being cultured in a suitable medium to increase the number of B. pseudomallei available and so to the genetic material associated thereto prior to reacting with the assembled CRISPR-based RNP system. The cultured and isolated bacteria of B. pseudomallei from the patient or the subject can be the biological sample in some embodiments of the disclosed method. For more embodiments, the biological sample may be a bodily fluid such as blood harvested from the subject or patient containing sufficient number or amount of the genetic materials of B. pseudomallei for a reaction with the established CRISPR-based RNP system using the disclosed crRNA.

The disclosed method may further comprise the step of amplifying segments of nucleotide sequence complementary to SEQ ID No. 3, SEQ ID No. 5 and SEQ ID No. 7 from the genetic materials to generate a plurality of amplicons; and reacting the amplicons with the assembled CRISPR-based RNP system along a plurality of signaling probes in a buffer solution instead of using the genetic materials directly in some of the embodiments. Despite these embodiments may incur extra time or cost compared to directly using the genetic materials of B. pseudomallei contained in the biological sample, the additional steps can become handy when the amount of genetic materials of B. pseudomallei is relatively low especially with limited biological sample obtained. Particularly, a segment in the genetic materials containing the CRISPR-based RNP system target site in the pathogen's genomic DNA is pre-amplified by flanking primers in an isothermal amplification reaction such as recombinase polymerase amplification (RPA), loop-mediated isothermal amplification (LAMP), or other thermocycling processes known in the field.

According to a number of the preferred embodiments of the disclosed method, the crRNA further comprises a repeat region preceding the spacer region forming a secondary structure thereof. More preferably, the repeat region comprises a polynucleotide sequence of SEQ ID No. 9 or SEQ ID No. 10. Furthermore, in some embodiments, the crRNA comprises a second extension region having a sequence of GGG being arranged preceding of the repeat region.

Pursuant to more embodiments of the disclosed method, the crRNA further comprises a first extension region arranged immediately after the spacer region. The first extension region shall comprise a polynucleotide sequence of AAAGGAA. As described earlier, addition of the first extension to the crRNA will result in better collateral cleavage activities towards the signalling probes thus enhancing detectability of signals released upon recognizing presence of the B. pseudomallei genetic materials in the biological sample.

In some embodiments of the disclosed method, the CRISPR-based RNP system is a CRISPR-Cas12a RNP system or a a CRISPR-Cas12a RNP complex. The Cas12a ribonuclease is an RNA-guide endonuclease having a capacity of collateral cleavage once activated and within an environment provided with single-stranded nucleic acid, ssDNA, used as part of the signalling or reporter probes of the disclosed method.

It is crucial to note that the plurality of signaling or reporter probes of the disclosed method is ssDNA or single-stranded polynucleotide with a detectable moiety and a corresponding quencher respectively attached at 5′-end and 3′-end of the single-stranded probe. The detectable moiety can be 5′6-fluorescein (FAM), Cy 3, HEX (Hexachloro-Fluorescein) or ROX (6-Carboxyl-X-Rhodamine) though FAM is employed in those more preferred embodiments. The quencher is preferably nonfluorescent molecules such as IABKFQ, DABCYL or BHQ being fashioned to suppress signal released from the detectable moiety unless the quenchers and the detectable moieties are removed from the ssDNA or ssRNA by way of collateral cleavage of the activated CRISPR-based RNP system upon recognizing the crRNA complementary sequences in the genetic materials. Particularly, the B. pseudomallei genetic materials of sufficient amount added to the assembled CRISPR-Cas12a RNP complex in a CRISPR reaction and the FAM-Quencher ssDNA probes (F-Q) will lead to recognition of the target sequences by the assembled CRISPR-RNP complex and the CRISPR-RNP complex self-activates its trans cleavage (collateral cleavage) activity to cut the signaling probes. This further results in cleavage of the FAM fluorophore from its quencher, hence the generation of a fluorescent signal, which can be detected by a fluorometer or the like allowing the disclosed method to confirm presence of B. pseudomallei genetic materials in the biological sample.

Moreover, further embodiments of the disclosed method preferably obviate the the requirement of an expensive laboratory setup like fluorometer. Preferably, the disclosed method further comprises the step of subjecting portion of the reacted buffer solution to a lateral flow assay for capturing the emitted signals. In order to realize the signal visualization through the lateral flow assay, minor changes have to be made towards the signaling or reporter probes. In some embodiments, the quencher is replaced by biotin to attain signal visualization. The signaling or reporter probes to be used for the lateral flow assay are preferably FAM-Biotin ssDNA. In more details, the lateral flow assay is a lateral flow dipstick generally comprising an absorbent platform on which carrying a sample pad section, a positive testing section or line, and a control section (or internal control line). Preferably, the sample pad section is impregnated with gold-conjugated anti-FAM antibodies. The control section is a section impregnated with streptavidin while the positive testing section is embedded with anti-IgG. It is worthwhile to note that the gold conjugate capable of chemically binding onto the FAM via the anti-FAM antibody is the reagent attributing to the visual signal observable at the positive and/or control section once enough amount of gold conjugates has been accumulated at these sections.

In practice, the disclosed method has the sample pad of a lateral flow dipstick directly immersed into the buffer solution containing the end products of the CRISPR reaction. This sample pad contains gold-conjugated anti-FAM antibodies. In the absence of B. pseudomallei genetic materials, the signalling probes remain intact and the gold conjugates binding to the FAM becomes trapped at the control section only due to the reaction between the impregnated streptavidin and the biotin on the intact signalling probe. Hence, only a single band can be seen at the control section. On the other hand, in the presence of the B. pseudomallei genetic materials, collateral activity of CRISPR-Cas12a degrades the probes, liberating the FAM-gold-conjugated-antibody complexes from biotin. The liberated FAM-gold-conjugated-antibody can diffuse passed the control section free from being entrapped by the streptavidin until reaching the positive testing section and being bound to the anti-IgG coated thereby giving rise to the visual cue indicating presence of the B. pseudomallei genetic materials in the biological sample. More specifically, two lines, one at the positive testing section and one at the control section (as not all reporter probes will be cleaved), can be sighted if the biological sample contains genetic materials of B. pseudomallei. Incorporating the lateral flow assay in the disclosed method permits a user to read the results of the CRISPR reaction free from the need of additional apparatus such as fluorometer and the like.

As mentioned in the setting forth, the disclosed method further verifies the genetic materials used are indeed originated from the B. pseudomallei instead of other pathogen sharing similar genetic makeup. Accordingly, the spacer region is being configured to hybridize on a complementary sequence resided in the genetic materials and the complementary sequence is preceded with a protospacer adjacent motif (PAM) comprising a sequence of TTTV with V being a non-T nucleotide.

It is worthwhile to note that the inventors of the present disclosure have developed a method highly sensitive and specific towards the detection of B. pseudomallei genomic DNA in a biological sample using CRIPSR RNP system, preferably a CRISPR-Cas12a. The presently developed detection platform is highly specific for B. pseudomallei as it detected all clinical isolates of endemic B. pseudomallei, while discriminating against human and other pathogens including its closely related species B. thailandensis as proven in the examples given hereinafter.

The adaptation of a lateral flow dipstick or assay in the disclosed method for a readout enabled the disclosed method, as a detection platform, to be performed in a regular laboratory, without the requirement of specific instruments. The disclosed method, particularly using along with the lateral flow assay, will facilitate point-of-care or field-deployable diagnosis towards B. pseudomallei.

### Example 1

pMBP-LbCas12a (from Lachnospiraceae bacterium species) was a gift from Jennifer Doudna (Addgene, USA, #113431). The plasmid was transformed into E. coli Rosetta 2 (DE3) (Nova-gen, USA, #70954). 500-mL of Luria-Bertani (LB) culture was grown at 37° C. until O.D.600 reached 0.4-0.6. MBP-LbCas12a expression was induced by growing the culture in the presence of 0.1 mM IPTG overnight at 16-18° C. Bacterial cells were harvested by centrifugation, washed with PBS and resuspended in 40 mL a lysis buffer (1000 mM NaCl, 20 mM imidazole, 20 mM Tris-HCl, 5% glycerol, 1 mM DTT and 1 mM PMSF). The cells were sonicated in an ice bath using Vibra-Cell VCX 500 sonicator (Sonics & Materials, USA) at a frequency 20 kHz with 60% amplitude for 3 minutes, using 1-second pulse, 7-second rest. Cell lysate was cleared by centrifugation at 20,000 g at 4° C. for 1 hour and filtered through a 0.22 μm PES membrane. All protein purification steps were performed using the AKTA Pure 25 M1 FPLC system (GE Healthcare, Sweden). First, the protein was affinity-purified on a 5-mL HisTrap HP column (GE Healthcare, Sweden, #17524802) with a His binding buffer (20 mM Tris-HCl pH 7.5, 1000 mM NaCl, 5% glycerol, 1 mM DTT, 20 mM imidazole), washed with a washing buffer (20 mM Tris-HCl pH 7.5, 1000 mM NaCl, 5% glycerol, 1 mM DTT, 50 mM imidazole) and eluted with an elution buffer (20 mM Tris-HCl pH 7.5, 1000 mM NaCl, 5% glycerol, 1 mM DTT, 300 mM imidazole). Peak fractions were collected and dialyzed overnight in a dialysis buffer (20 mM Tris-HCl pH 7.5, 125 mM NaCl, 5% glycerol, 1 mM DTT, 1 mM PMSF). Soluble protein was recovered and loaded into a 5-mL HiTrap SP HP cation exchange column (GE Healthcare, Sweden, #17115201), pre-equilibrated with a binding buffer (20 mM Tris-HCl pH 7.5, 125 mM NaCl, 5% glycerol). The column was washed with the binding buffer, and protein was eluted with an ion exchange buffer (20 mM Tris-HCl pH 7.5, 5% glycerol, 125-2000 mM NaCl gradient). Fractions containing MBP-LbCas12a were combined, concentrated and injected into a HiLoad 16/600 Superdex 200 pg size exclusion column (GE Healthcare, Sweden, #28989335), pre-equilibrated with a gel filtration buffer (50 mM Tris-HCl pH 7.5, 500 mM NaCl, 5% glycerol). Protein was eluted with the same buffer. Fractions containing MBP-LbCas12a were concentrated to ˜500 μl. Glycerol and DTT were added to achieve final concentrations of 20% v/v and 2 mM, respectively. The protein was aliquoted, snap frozen in liquid nitrogen and stored at −80° C.

### Example 2

All command codes used were open-source at GitHub (https://github.com/henrikroslund/crispr-cas12a/releases/tag/BP_V2). In the first step, CRISPR selection, one of 30 representatives B. pseudomallei genome, namely bb2, was used to generate an initial pool of all possible LbCas12a target sites by scanning for TTTVN₁ . . . . N₂₀ (PAM is indicated in bold; Vis non-T nucleotide, and N1 to N20 is spacer sequence of any nucleotides). Duplicate target sites or target sites with spacer regions that contained quadruple nucleotides or GC content outside 40-65% were removed. This resulted in 26,661 candidate target sites, which were subsequently aligned to the other 29 representative B. pseudomallei genomes. 15,448 candidate target sites were found to be 100% conserved in all 30 genomes and were processed to the CRISPR filtering step to remove potential cross reactions with other pathogens. First, a pool of CRISPR-Cas12a target sites TTTNN₁ . . . . N₂₀ from 1,071 complete genomes of non-B. pseudomallei pathogens was generated. The 4th position of PAM was relaxed, so that weaker cross-reactive target sites were generated. The candidate target sites from B. pseudomallei were aligned to target sites from non-B. pseudomallei pathogens using only N₁ to N₂₀, and candidates with 100% match were removed. This initial filtering step significantly trimmed down the number of B. pseudomallei candidate target sites to 1,982. The resulting candidates were further filtered out by re-mapping to the above pool of target sites from non-B. pseudomallei pathogens. Mappability was defined as having at least 15 nucleotides matching in N₁ to N₂₀. Mapped candidates were analyzed for mismatches relative to their cross-reactive target sites. Candidates that failed to fulfil the following ‘mismatch criteria’ at any mapped sites were immediately removed: (i) 2 consecutives in Seed (N₁ to N₆), or (ii) 3 or more in Seed, or (iii) 4 or more in N₇ to N₂₀. The remaining candidates and candidates that were unable to be mapped to any cross-reactive target sites were considered as the final candidates (Table 1). Spacer regions (excluding PAM) were later engineered into crRNA.

TABLE 1

| | | Copy | | | | % |
|---|---|---|---|---|---|---|
| Name | Target sequences | No. | Location | Annotation | Product | Coverage |
| crBP32 | TTTATCGTAACATAATTGCCGGGC | 1 | Chr2: | BPSS2080 | Hypo- | 98.05 |
| | | | 2,814,418- | | thetical | (3276/3341) |
| | | | 2,814,441 | | protein | |
| crBP33 | TTTGGACGATCGCCTCCGTTCTTG | 1 | Chr2: | Non- | — | 99.94 |
| | | | 2,373,476- | coding | | (3339/3341) |
| | | | 2,373,499 | | | |
| crBP34 | TTTAACTACATACCCACTATTCAG | 1 | Chr2: | Non- | — | 99.82 |
| | | | 897,403- | coding | | (3335/3341) |
| | | | 897,380 | | | |
| crBP35 | TTTGCCGCCACCCAATCCGAGTCA | 1 | Chr2: | BPSS0448 | Hypo- | 99.82 |
| | | | 613,529- | | thetical | (3335/3341) |
| | | | 613,552 | | protein | |
| crBP36 | TTTGAGTTCGAGGCGGTCCGAAGC | 1 | Chr2: | BPSS1386 | ATP/GTP | 99.43 |
| | | | 1,899,187- | (BepE) | binding | (3322/3341) |
| | | | 1,899,164 | | protein; | |
| | | | | | T3SS-1 | |
| crBP37 | TTTCGTGCGCGAAGTTATGTGGCA | 1 | Chr2: | BPSS2085 | ABC | 98.41 |
| | | | 2,821,949- | | transporter | (3288/3341) |
| | | | 2,821,926 | | ATP- | |
| | | | | | binding | |
| | | | | | protein | |
| crBP38 | TTTAATCTCAAGACGACGTCCAAT | 1 | Chr1: | Non- | — | 97.93 |
| | | | 1,950,016- | coding | | (3272/3341) |
| | | | 1,949,993 | | | |
| crBP39 | TTTCAATGGTCGCGAACCCACCGT | 1 | Chr2: | Non- | — | 99.91 |
| | | | 1,643,228- | coding | | (3338/3341) |
| | | | 1,643,251 | | | |

### Example 3

DNA templates were synthesized and desalted by IDT DNA technologies (Singapore). All crRNAs were in vitro transcribed from annealed oligo templates using a HiScribe T7 High Yield RNA Synthesis Kit (NEB, USA, #E2040S), in accordance with the manufacturer's protocol. DNAse I (RNase-free) (NEB, USA, #M0303S) was added to the reaction, and the tube was incubated at 37° C. for 20 minutes to enable the degradation of the DNA template. crRNAs were purified in 12% v/v urea-PAGE (29:1) (in 7.5 M urea, 0.5×TBE), eluted in a gel elution buffer (300 mM sodium acetate pH 5.2, 1 mM EDTA pH 8.0, 0.1% v/v SDS) and precipitated in ethanol. crRNA was resuspended in a folding buffer (10 mM Tris-HCl pH 7.5, 50 mM NaCl), folded by heating in a metal heat block at 90° C. for 2 minutes and allowed to slowly cool down at room temperature. crRNA was aliquoted and stored at −80° C. until used.

### Example 4

A global collection of B. pseudomallei (n=3,341) was compiled from the public database and other short-read sequenced data. When the assemblies were not available, short reads were de novo assembled using Velvet, giving a median of 136 contigs (range 60-434), and an average total length of 7,140,818 bps (min=6,989,389 bps and max=7,441,976 bps). Gene predictions and annotations of assemblies were performed using Prokka. With an average of 5,850 (range 5685-6275 per each genome) predicted coding sequences (CDS) assigned, the assemblies fall in a similar range of 6,332 CDS and length of 7,247,547 bps as reported in the reference genome K96243. Population structure was defined using three independent approaches-PopPUNK, a phylogenetic tree, and multi-dimensional scaling as described previously. All three methods showed high consistency and resolved the B. pseudomallei population into 324 lineages, many of which are singletons or lineages with a small number of isolates. The present disclosure noted that there are 22 major lineages dominating the global B. pseudomallei population (2,429/3,341 isolates representing 73% of the dataset).

To ensure that the CRISPR-Cas targets cover the genetic diversity of the entire population, candidate genomes were selected to represent each major lineage (Burkholderia Bayesian cluster (bb) 1 to 22 (FIG. 1B), except for bb3 and bb4 which shared close ancestry with bb5 where one representative was selected to represent three lineages. Inventors of the present disclosure also randomly selected candidate genomes outside the major lineages. Collectively, a total of 30 genomes were selected. On average, each candidate could be assembled into 92 contigs, thereby enabling a fair length of conserved regions for the searching of CRISPR-Cas targets. The PopPUNK phylogenetic tree (FIG. 1B) was visualized and annotated with the metadata using Tree of Life Tools.

### Example 5

An in silico validation of CRISPR-Cas targets were performed by mapping the designed RPA primers and target sequences for CRISPR-Cas12a against the assemblies of 3,341 B. pseudo-mallei genomes (S1 Table) using BLAT v. 36. To enable mapping of sequences with low com-plexity, the command “blat-minScore=10-tileSize=8<assembly><candidate target><out.psl>” was employed. As CRISPR-Cas12a recognition seems to be flexible at the 4th position of PAM and seem to allow one sequence mismatch in the position N₁ to N₂₀ (though the activity is reduced), the present disclosure further conditioned the BLAT results to allow for two types of mismatches. For the sequence target TTTVN₁N₂N₃ . . . . N₂₀, the analysis allowed for (i) a mismatch at position V; or (ii) a mismatch at any of the position from N₁ to N₂₀; or a combination of (i) and (ii). To test for possible cross-reactivity of these CRISPR-Cas12a target sequences with human host DNA or other bacterial species, inventors additionally mapped them against a human reference genome GRCh38 and 40,827 bacterial reference genomes archived in NCBI repository (date retrieved 5 May 2022). To reduce computational load, the search was performed using NCBI web BLASTN with results filtered to allow for (i) a mismatch at position V; or (ii) a mismatch at any of the position from N₁ to N₂₀; or a combination of both as outlined earlier. None of the hits passed the filtering threshold and are unlikely to be recognized by CRISPR Cas12a, given that a single mismatch in N₁ to N₂₀ already significantly reduces the enzyme's function.

CRISPR-Cas12a is an RNA guided-ribonucleoprotein (RNP) complex that recognizes its target DNA through base-pairing complementarity [37,38]. Recognition and cleavage of the target transform the RNP complex into a non-specific nuclease that collaterally cleaves single-stranded DNA (ssDNA) in vitro [29]. This collateral cleavage property and programmability of CRISPR-Cas12a offer an adaptation for highly specific and sensitive detection of DNA [29,31]. Therefore, the present disclosure aimed to apply this CRISPR diagnostic platform to the detection of B. pseudomallei DNA.

A key to the specificity of CRISPR-Cas12a-based detection is a spacer region of the crRNA, which base-pairs to target DNA (FIG. 1A). Since B. pseudomallei is highly recombinogenic with constant reshuffling of genomic contents [39-41], a well-designed spacer to target a DNA sequence that is present in all B. pseudomallei is needed to ensure high coverage of detection. Thus, the present disclosure performed a phylogenetic analysis of 3,341 B. pseudomallei genomes from a global collection. The analysis classified the B. pseudomallei population into 22 major lineages (FIG. 1B). The present disclosure then selected a representative from each major lineage and eight other outsider representatives for our in-house bioinformatic pipeline, which searches for CRISPR-Cas12a candidate target sites (FIG. 1C).

In this pipeline, inventors of the present disclosure first generated a pool of optimal target sites that are common among the 30 B. pseudomallei representative genomes. These target sites contain TTTV (V is A, C or G) as a protospacer adjacent motif (PAM) on 5′ end followed by a 20-nt long spacer (FIG. 1A). The resulting candidate target sites were then filtered through 1,071 genomes of various bacterial pathogens that cause frequent infections or those that are closely related to B. pseudomallei including B. cepacia, B. mallei and B. thailandensis. This final step yielded candidate sites that either are unique to B. pseudomallei or contain significant mismatches to those cross-reactive pathogens; both of which could be highly specific target sites for CRISPR-Cas12a. The results showed that after executing the bioinformatic pipeline, the inventors retrieved eight elite target sites, each found as a single copy in a B. pseudomallei genome (Table 1). These target sites are located in both coding and non-coding regions of the genome. Using in silico search for potential cross-reactive sites against the NCBI genomic database (data retrieved on May 5, 2022), the present disclosure further confirmed a high specificity of all target sites with no cross-reaction with human or other bacterial genomes [36]. In contrast, when analyzing coverage of these eight target sites on a B. pseudomallei global population, it was found that all of them can be found in at least in 97% of the population (Table 1).

### Example 6

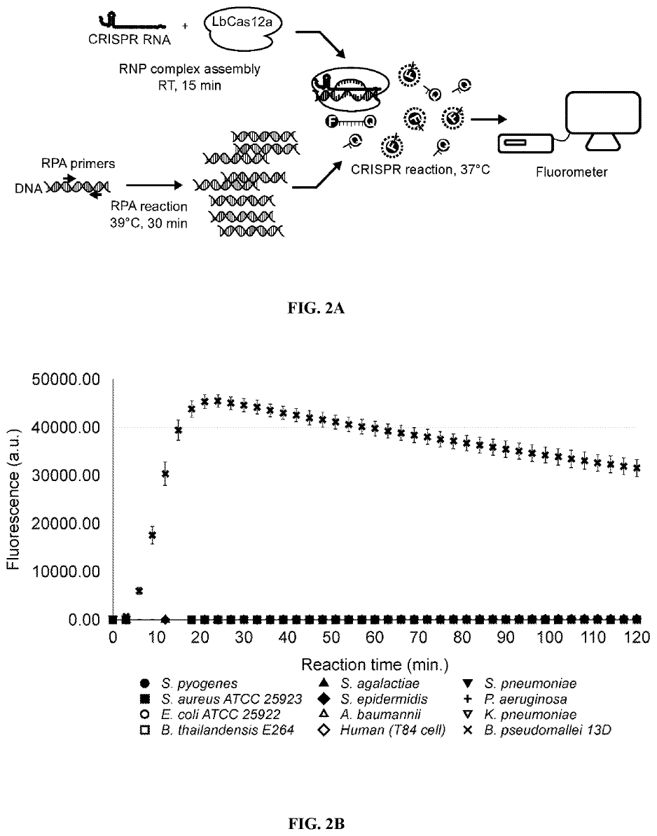

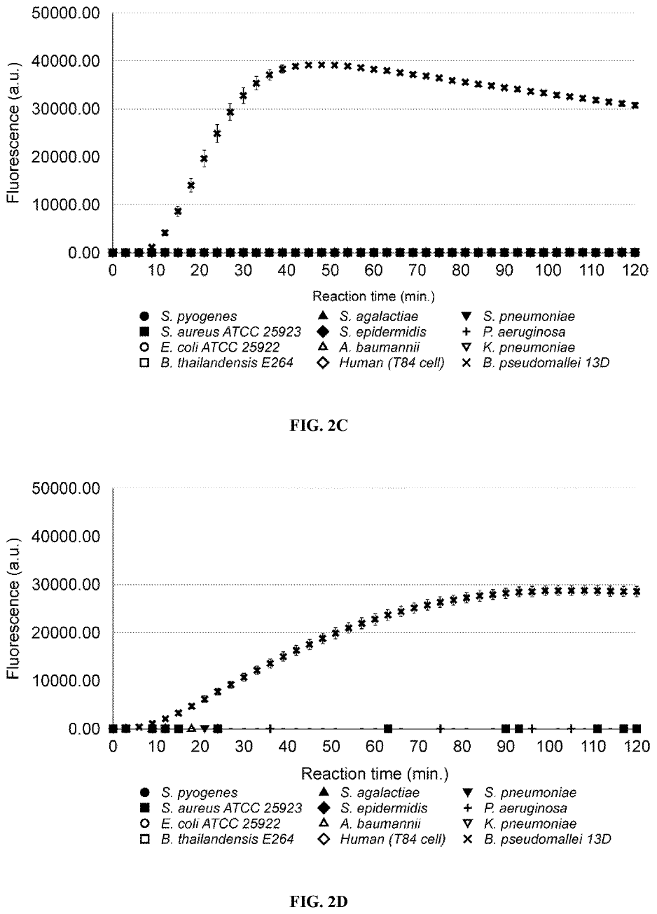

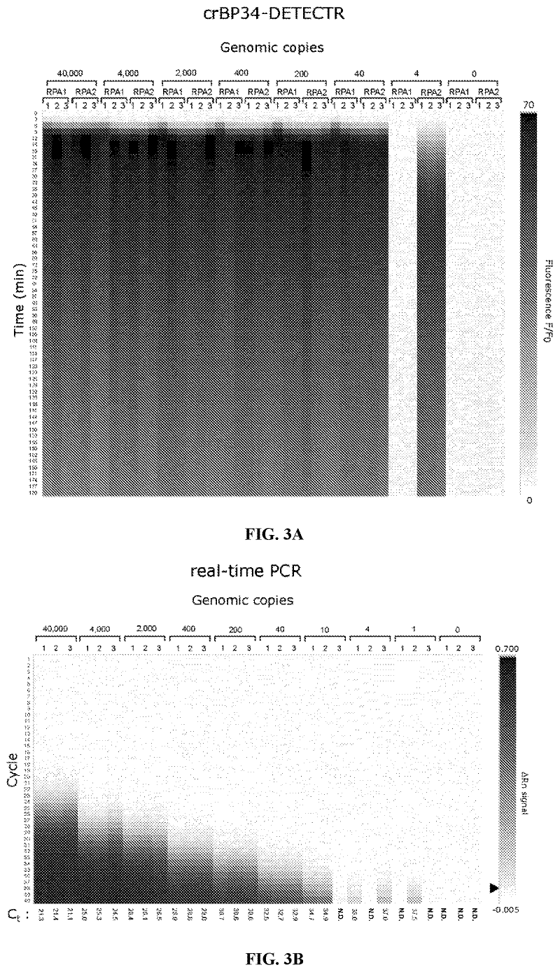

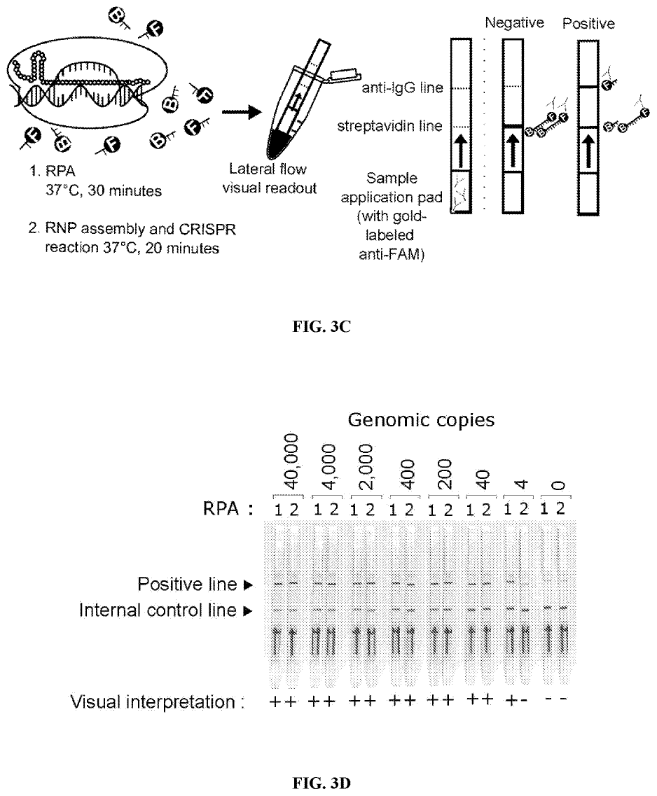

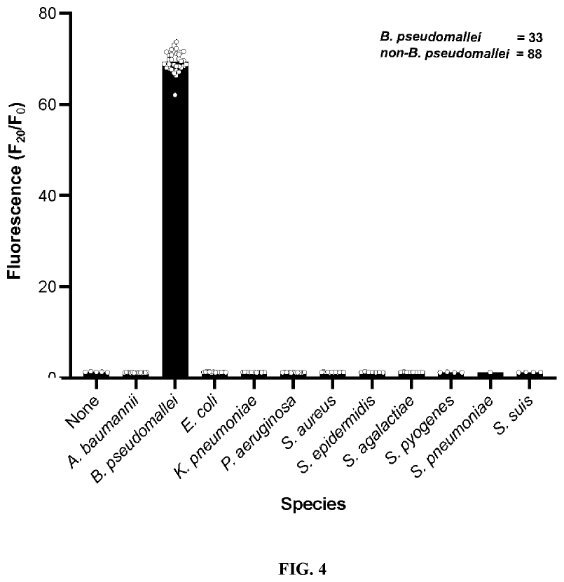

RPA reagent was purchased from TwistDx (USA). All RPA primers were 35-nt long with amplicon length less than 500 base-pairs (ideally 100-200 base-pairs), as recommended by TwistDx. These primers were manually designed and checked for their secondary structures and homo-/hetero-duplex using RNAfold (http://rna.tbi.univie.ac.at/cgi-bin/RNAWebSuite/RNAfold.cgi) and IDT OligoAnalyzer (https://www.idtdna.com/pages/tools/oligoanalyzer), respectively. Primers were synthesized and purified by standard desalting by Macrogen (Korea). TwistAmp Basic (TwistDx, USA, #TABAS03KIT) and TwistAmp Liquid Basic (TwistDx, USA, #TALQBAS01) were both used in the present study in accordance with the manufacturer's protocols, with the following modifications: (i) total volume of an RPA reaction was adjusted to 30-50 μl and (ii) incubation was done at 39° C. for 30 minutes. For the specificity test (FIGS. 2B, 2C and 2D), ˜10,000 copies of bacterial genomic DNA were used in each reaction. The copy number was approximated from the median genomic size of each bacterial species. For the sensitivity test (FIGS. 3A and 3B), the accurate copy number of B. psuedomallei (isolate 13D) genomic DNA stock was determined by real-time PCR against purified standards. The genomic DNA was then diluted to appropriate concentrations and immediately used in the RPA reaction. For detection of clinical isolates (FIG. 4), 1 ng of DNA was used per RPA reaction. All RPA reactions were stored at −20° C. until used.

### Example 7

Non-B. pseudomallei clinical isolates were obtained from Diagnostic Laboratory, Maharaj Nakorn Chiang Mai hospital. Colonies were directly scrapped off sheep-blood agar plates and resuspended in 200 μl water. A GeneJet genomic DNA purification kit (ThermoFisher Scientific, USA, #K0721) was used to extract genomic DNA in accordance with the manufacturer's protocols. B. pseudomallei clinical isolates were obtained from 33 cases of melioidosis patients admitted to Sunpasitthiprasong hospital in Ubon Ratchathani, Thailand. Samples came from either blood, urine, or throat swabs of melioidosis patients and cultured on Ashdown's agar to select for the growth of B. pseudomallei. For each sample, a single colony was picked and streaked on a Columbia agar and incubated at 37° C. overnight. A loop full of B. pseudomallei from the Columbia agar was then inoculated in 3 mL Luria-Bertani broth and incubated at 37° C. with shaking at 200 rpm for 16-18 hours in the biosafety level 3 laboratory. B. pseudomallei was harvested and lysed for genomic DNA extraction using QIAmp Mini kit protocol (QIAGEN, Germany, #51304).

### Example 8

CRISPR reactions were performed in a final 100-μl volume that contained 100 nM crRNA, 200 nM MBP-LbCas12a, 500 nM FAM-Quencher probe and 1×CR buffer 1 (10 Tris-HCl pH 8.0, 50 mM NaCl, 13 mM MgCl2, 1% v/v glycerol). First, CRISPR-Cas12a ribonucleoprotein (RNP) was pre-assembled in 30-μl volume with crRNA, MBP-LbCas12a and CR buffer 1. The reaction was incubated at room temperature for 15 minutes. A 70-μl mixture of FAM-Quencher probe with various amounts of RPA was added to the RNP. This CRISPR reaction was transferred to a fluorescence plate reader Synergy H4 (BioTek, USA) and read at 483/530 nm (excitation/emission) every 3 minutes for 3 hours at 37° C.

To verify that the identified target sites can be used with CRISPR-Cas12a to detect B. pseudomallei genomic DNA, the present disclosure engineered CRISPR-Cas12a to contain target sites in the spacer region of crRNA and applied these modified CRISPR-Cas12a enzyme to DETECTR assay (FIG. 2A). In the DETECTR assay, the pathogen's DNA is pre-amplified by an isothermal method before being combined with pre-assembled CRISPR-Cas12a RNP in a CRISPR reaction [29]. This reaction contains an excess amount of short ssDNA probes modified with fluorescein and quencher on 5′ and 3′ ends (FAM-Quencher probe), respectively. In the presence of the pathogen's DNA, a programmed CRISPR-Cas12a RNP complex recognizes an intended target and activates itself into a non-specific ssDNAse that cleaves the probes in trans. Liberation of the fluorescein from the quencher results in an emission of fluorescence, a proxy for detection of the pathogen's DNA.

The present disclosure designed 35-nt primers and performed RPA on only seven target sites. The exception was crBP33 of which optimal primers could not be designed due to highly repetitive sequences and GC-rich nature in this region. The result showed that RPA of target sites crBP34, crBP36 and crBP38 yielded good amplification products among the seven target sites. Therefore, the present disclosure selected these candidate target sites and engineered their 20-nt sequences (excluding PAM) into the spacer region of the crRNA (FIG. 1A). The 7-nt RNA extension was also added to the 3′ end of the crRNA, as it has been shown to increase collateral cleavage activity [43]. Genomic DNA from 11 pathogens that are common causes of bacterial infections in melioidosis endemic areas, including S. pyogenes, S. epidermidis, K. pneumoniae, S. agalactiae, P. aeruginosa, B. thailandensis, S. pneumoniae, E. coli, S. aureus, A. baumannii and B. pseudomallei were pre-amplified using RPA and tested with each crRNA. The results of fluorescence activation showed that all three crRNAs could specifically detect B. pseudomallei, but not other pathogens (FIGS. 2B, 2C and 2D). Encouragingly, all three crRNAs could discriminate effectively against B. pseudomallei's close relative B. thailandensis. This specificity exceeded expectation since fluorescent signals from non-B. pseudomallei bacteria were barely above the background level, in contrast to that from B. pseudomallei which exhibited 4-order of magnitude of intensity. Since there is the potential to use this platform to diagnose melioidosis in humans, the present disclosure also tested crBP34, 36 and 38 with human genomic DNA. Inventors found that all three crRNAs did not cross-react with human DNA (FIGS. 2B, 2C and 2D). These results validated the crRNA design pipeline that excluded cross-reaction from non-B. pseudomallei DNA (FIG. 1A). In addition, the present disclosure noticed that each crRNA exhibited differing kinetics of detection. crBP34 had the strongest signal that reached a maximum at 24 minutes, while crBP36 reached a maximum at 48 minutes. crBP38, on the other hand, displayed the most delayed maximum at 108 minutes. These variations could be a result of cumulative differences in both RPA efficiency and the intrinsic property of each crRNA. Regardless, all three crRNAs displayed specific signals above the background in less than 10 minutes into the reaction. Since crBP34 possesses the strongest signal and the fastest kinetics, inventors selected crBP34 for the subsequent experiments detailed in example 9.

### Example 9

RNP complex assembly and CRISPR reactions were combined and performed in a final 50-μl volume that contained 1×CR buffer 1, 100 nM crRNA, 200 nM MBP-LbCas12a, 100 nMFAM-Biotin probe and 5 μl RPA. The reaction was incubated at 37° C. for 20 minutes. A Hybri-Detect lateral flow dipstick (Milenia Biotek, Germany, #MGHD1) was directly dipped into the reaction and allowed to develop for 2-5 minutes.

Real-time PCR primer were reported previously [17]. The primers were synthesized and desalted by Macrogen (South Korea). 20-μl real-time PCR reactions were prepared using Maxima SYBR Green/ROX qPCR Master Mix (ThermoFisher Scientific, USA, #K0221) in accordance with the manufacturer's instruction. Real-time PCR analysis was performed on the 7500 Fast Real-Time PCR System (Applied Biosystems) with the following conditions: 95° C. (10 minutes); 40 cycles of 95° C. (15 seconds), 61° C. (30 seconds), 72° C. (30 seconds). Standard melting curve analysis was also performed at the end of the PCR. ΔRn threshold was automatically calculated by the built-in program.

One important feature of a reliable diagnostic tool is sensitivity. To investigate the ability of crBP34-coupled DETECTR in detecting trace amounts of genetic material, frequently present in clinical samples, the present disclosure determined the limit of detection (LOD) of this CRISPR platform. A known copy number of genomic DNA of B. pseudomallei was serially diluted and used in the assay as described above. The result showed that the developed CRISPR-Cas12a detection platform has an LOD at 40 copies of B. pseudomallei genomic DNA per reaction (FIG. 3A). In fact, the assay could detect as low as four copies of input genomic DNA; however, at this lower DNA amount, detection was inconsistent, and the fluorescent signal was weaker and had a more delayed rate of detection (maximum at 55 minutes). For comparison, the present disclosure also performed real-time PCR—a sensitive standardized method for nucleic acid detection. When comparing the LOD of crBP34-DETECTR to that of real-time PCR, it was found that the developed detection platform performed equally well as did this standard method, which showed reliable detection at 40 copies per reaction (FIG. 3B). In addition, the inventors tested this detection platform with B. pseudomallei genomic DNA spiked with different amounts of human genomic DNA. As expected, the result showed that the sensitivity was negatively impacted by the presence of human DNA background, especially at LOD of the assay.

To obviate the requirement of an expensive laboratory setup, the inventors of the present disclosure replaced detection of fluorescence with a lateral flow dipstick for the assay readout. Consequently, the FAM-Quencher ssDNA probes were substituted by FAM-Biotin ssDNA probes in the CRISPR reaction. Afterwards, a sample pad of a lateral flow dipstick was directly immersed into the CRISPR reaction. This sample pad contains gold-conjugated anti-FAM antibodies, which label FAM moieties for visualization-both in intact probes and in cleaved probes (FIG. 3C). In the absence of B. pseudomallei target DNA, the probes are intact and trapped at the streptavidin line by biotin-streptavidin interaction. However, in the presence of the target DNA, collateral activity of CRISPR-Cas12a degrades the probes, liberating the FAM-antibody complexes from biotin so that they can diffuse further on the dipstick to bind to the anti-IgG line. The present disclosure tested this readout with various amounts of B. pseudomallei genomic DNA and found that the sensitivity of this readout correlates well with that of the fluorescence readout, with an LOD at 40 copies per reaction (FIG. 3D). Therefore, the lateral flow dipstick can be used to give a sensitive readout of the assay, providing an easy method for detection of B. pseudomallei.

In silico analysis indicated that all eight identified CRISPR-Cas12a target sites were highly conserved. crBP34 has sequences that match 3,335 out of 3,341 B. pseudomallei genomes (Table 1) and were shown to be very specific in the preliminary data (FIG. 2B). To verify that the presently designed crBP34 can specifically detect other B. pseudomallei isolates, the present disclosure tested the developed CRISPR diagnostic platform with various clinically isolated bacteria, specifically B. pseudomallei and common non-B. pseudomallei pathogens. The results showed that crBP34 could accurately detect all B. pseudomallei isolates (n=33) (FIG. 4). Importantly, the assay discriminated against all other DNA from the non-B. pseudomallei pathogens (n=88). The fluorescence signals from this latter group of pathogens remained at a baseline, at the same level of the background (water-input RPA, ‘None’). Together, these data suggest that crBP34 exhibits 100% specificity and could potentially be used to detect B. pseudomallei from different geographical areas. Table 2 below further illustrates descriptive statistic data obtained with respect to the various clinical bacterial isolates tested using the develop crBP34-DETECTR (or SEQ ID No.3-DETECTR).

TABLE 2

| | | Fluorescent signal (at 20 min/0 min) | | | | |
|---|---|---|---|---|---|---|
| | | | | | | Std. |
| | | | | | | Devi- |
| Species | N | Minimum | Maximum | Range | Mean | ation |
| None | 5 | 1.195 | 1.443 | 0.25 | 1.306 | 0.089 |
| A. baumannii | 15 | 1.096 | 1.269 | 0.17 | 1.169 | 0.047 |
| B. pseudomallei | 33 | 62.087 | 73.728 | 11.64 | 69.37 | 2.251 |
| E. coli | 12 | 1.155 | 1.322 | 0.17 | 1.247 | 0.056 |
| K. pneumoniae | 14 | 1.146 | 1.284 | 0.14 | 1.224 | 0.041 |
| P. aeruginosa | 11 | 1.115 | 1.286 | 0.17 | 1.211 | 0.049 |
| S. agalactiae | 8 | 1.236 | 1.357 | 0.27 | 1.262 | 0.041 |
| S. aureus | 12 | 1.126 | 1.397 | 0.18 | 1.235 | 0.062 |
| S. epidermidis | 7 | 1.201 | 1.377 | 0.12 | 1.251 | 0.059 |
| S. pyogenes | 4 | 1.189 | 1.307 | 0.12 | 1.228 | 0.054 |
| S. pneumoniae | 1 | 1.236 | 1.236 | NA | 1.236 | NA |
| S. suis | 4 | 1.195 | 1.295 | 0.1 | 1.25 | 0.041 |

### Example 10

Nucleic acid detection by isothermal amplification assays such as RPA and LAMP have had increased usages in research communities due to their high sensitivity and simplicity. Nevertheless, LAMP demands a series of six to eight primers to work together in an optimal locus [44]. This requirement could possibly limit its broad usage in the detection of B. pseudomallei, which is heterogenous and GC-rich in its genome. RPA, on the other hand, can tolerate significant mismatches in primers [32], making it less specific. Inventors often observed non-specific amplification in the RPA reactions performed. In comparison, the DETECTR assay combines RPA and CRISPR-Cas12a. It utilizes CRISPR-Cas12a as a sequence-specific DNA reader to verify the identity of pre-amplified DNA from RPA reaction. This adds an extra stringency to RPA while maintaining its high sensitivity. As a result, the develop method or assay is highly specific and sensitive. The assay has an LOD at 40 copies per reaction, comparable to that of real-time PCR (FIG. 3B), LAMP and RPA [29,30].

The specificity of the crBP34-DETECTR may arise from the designed crRNA-crBP34, which was a result of large-scale phylogenetics and bioinformatics analyses to remove cross-reactivity (FIGS. 1B and 1C). This contrasts with previously reported detection assays using real-time PCR [21-25], LAMP [26,27] or RPA [28-30], in which primers were designed based on selected genomic loci or limited bioinformatics analysis on few reference strains. Testing crBP34 and the other crRNAs with B. pseudomallei isolates from other geographical areas remains for further examination to assess their universal application. However, the coverage analysis predicted that both the CRISPR-Cas12 target sites (Table 1) and RPA primers are present in more than 97% of the global B. pseudomallei population. This gives a high probability of detection by the designed crRNAs.

When mixing human DNA with B. pseudomallei DNA, inventors observed a reduction of the RPA product, resulting in a subsequent loss of collateral activity of the CRISPR-Cas12a. Hence, DNA extraction methodology that selectively enriches pathogens from clinical samples will play a crucial role in the detection

Aspects of particular embodiments of the present disclosure address at least one aspect, problem, limitation, and/or disadvantage associated with existing kit or method in relation to rapid and relatively easy detection of Burkholderia pseudomallei in a biological sample acquired from a human subject. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed structures, components, or alternatives thereof, can be desirably combined into alternative structures, components, and/or applications. In addition, various modifications, alterations, and/or improvements may be made to various embodiments that are disclosed by a person of ordinary skill in the art within the scope of the present disclosure, which is limited only by the following claims.

### REFERENCES

1. Gassiep I, et al. Human Melioidosis. Clin Microbiol Rev. 2020; 33: e00006-19.
2. Limmathurotsakul D, et al. Predicted global distribution of Burkholderia pseudomallei and burden of melioidosis. Nat Microbiol. 2016; 1:15008.
3. Chaowagul W, et al. Melioidosis: a major cause of community-acquired septicemia in northeastern Thailand. J Infect Dis. 1989; 159:890-899.
4. Chakravorty A and Heath C H. Melioidosis: An updated review. Aust J Gen Pract. 2019; 327-332.
5. Hinjoy S, et al. Melioidosis in Thailand: Present and Future. Trop Med Infect Dis. 2018; 3:38.
6. Cheng A C & Currie B J. Melioidosis: Epidemiology, Pathophysiology, and Management. Clin Microbiol Rev. 2005; 18:383-416.
7. Wiersinga W J, et al. Melioidosis. Nat Rev Dis Primers. 2018; 4:1-22.
8 Dance D. Treatment and prophylaxis of melioidosis. Int J Antimicrob Agents. 2014; 43:310-318.
9. Chaichana P, et al. Antibodies in Melioidosis: The Role of the Indirect Hemagglutination Assay in Evaluating Patients and Exposed Pop-ulations. Am J Trop Med Hyg. 2018; 99:1378-1385.
10. Houghton R L, et al. Development of a Prototype Lateral Flow Immunoassay (LFI) for the Rapid Diagnosis of Melioidosis. PLOS Neglected Tropical Dis-eases. 2014; 8: e2727.
11. Phokrai P, et al. A Rapid Immunochromatography Test Based on Hcp1 Is a Potential Point-of-Care Test for Sero-logical Diagnosis of Melioidosis. J Clin Microbiol. 2018; 56: e00346-18.
12. Robertson G, et al. Rapid diagnostics for melioidosis: a comparative study of a novel lateral flow antigen detection assay. J Med Microbiol. 2015; 64:845-848.
13. Suttisunhakul V, et al. Evaluation of Polysaccharide-Based Latex Agglutination Assays for the Rapid Detection of Antibodies to Burkholderia pseudomallei. Am J Trop Med Hyg. 2015; 93:542-546.
14. Wagner G E, et al. Melioidosis DS rapid test: A standardized serological dipstick assay with increased sensitivity and reliability due to multiplex detection. PLOS Neglected Tropical Diseases. 2020; 14: e0008452.
15. Woods K L, et al. Evaluation of a Rapid Diagnostic Test for Detection of Burkholderia pseudomallei in the Lao People's Democratic Republic. J Clin Microbiol. 2018; 56: e02002-17.
16. Yatsomboon A, et al. Development of an immunomagnetic separa-tion-ELISA for the detection of Burkholderia pseudomallei in blood samples. Asian Pac J Allergy Immunol. 2021; 39:35-43.
17. Novak R T, et al. Development and Evaluation of a Real-Time PCR Assay Targeting the Type III Secretion System of Burkholderia pseudomallei. J Clin Microbiol. 2006; 44:85-90.
18. Price E P, et al. Development and Validation of Burkholderia pseudomallei-Specific Real-Time PCR Assays for Clinical, Environmental or Forensic Detection Applications. PLOS ONE. 2012; 7: e37723.
19. Chantratita N, et al. Loop-mediated isothermal amplification method targeting the TTS1 gene cluster for detection of Bur-kholderia pseudomallei and diagnosis of melioidosis. J Clin Microbiol. 2008; 46:568-573.
20. Li J, et al. Preliminary Evaluation of Rapid Visual Iden-tification of Burkholderia pseudomallei Using a Newly Developed Lateral Flow Strip-Based Recombi-nase Polymerase Amplification (LF-RPA) System. Front Cell Infect Microbiol. 2022; 11:804737.
21. Peng Y, et al. Rapid detection of Burkholderia pseudomallei with a lateral flow recombinase polymerase amplification assay. PLOS ONE. 2019; 14: e0213416.
22. Hille F, et al. The Biology of CRISPR-Cas: Back-ward and Forward. Cell. 2018; 172:1239-1259.
23. Makarova K S, et al. SnapShot: Class 1 CRISPR-Cas Systems. Cell. 2017; 168:946-946.e1.
24. Makarova K S, et al. SnapShot: Class 2 CRISPR-Cas Systems. Cell. 2017; 168:328-328.e1.
25. Wang, J. Y. and Doudna, J. A. CRISPR technology: A decade of genome editing is only the beginning. Science 379, eadd8643 (2023).
26. Freije C A and Sabeti P C. Detect and destroy: CRISPR-based technologies for the response against viruses. Cell Host & Microbe. 2021; 29:689-703.
27. Mancuso P, et al. CRISPR based editing of SIV proviral DNA in ART treated non-human primates. Nat Commun. 2020; 11:6065.
28. Abudayyeh O O, et al. RNA targeting with CRISPR-Cas13. Nature. 2017; 550:280-284.
29. Chen J S, et al. CRISPR-Cas12a target binding unleashes indiscriminate single-stranded DNase activity. Science. 2018; 360:436-439.
30. Gootenberg J S, et al. Nucleic acid detection with CRISPR-Cas13a/C2c2. Science. 2017; 356:438-442.
31. Broughton J P, et al. CRISPR-Cas12-based detec-tion of SARS-COV-2. Nature Biotechnology. 2020; 38:870-874.
32. Kellner M J, et al. SHERLOCK: nucleic acid detection with CRISPR nucleases. Nature Protocols. 2019; 14:2986-3012.
33. Mustafa M I & Makhawi A M. SHERLOCK and DETECTR: CRISPR-Cas Systems as Potential Rapid Diagnostic Tools for Emerging Infectious Diseases. Journal of Clinical Microbiology. 2020 [cited 21 Jan. 2022].
34. Shi K, et al. A CRISPR-Cas autocatalysis-driven feedback amplification network for supersensitive DNA diagnostics. Science Advances. 2021; 7: eabc7802.
35. NCBI human genome. [cited 10 Jun. 2022].
36. NCBI bacterial genomes. [cited 10 Jun. 2022].
37. Dong D, et al. The crystal structure of Cpf1 in complex with CRISPR RNA. Nature. 2016; 532:522-526.
38. Yamano T, et al. Crystal Structure of Cpf1 in Complex with Guide RNA and Target DNA. Cell. 2016; 165:949-962.
39. Tuanyok A, et al. Genomic islands from five strains of Burkholderia pseudomallei. BMC Genomics. 2008; 9:566.
40. Tumapa S, et al. Burkholderia pseudomallei genome plasticity associated with genomic island variation. BMC Geno-mics. 2008; 9:190.
41. U'Ren J M, et al. Tandem repeat regions within the Burkholderia pseudomallei genome and their application for high resolution genotyping. BMC Microbiology. 2007; 7:23.
42. Sayers E W, et al. Database resources of the national center for biotechnology information. Nucleic Acids Res. 2022; 50: D20-D26.
43. Nguyen L T, et al. Enhancement of trans-cleavage activity of Cas12a with engineered crRNA enables amplified nucleic acid detection. Nature Communications. 2020; 11:4906.
44. Wong Y-P, et al. Loop-mediated isothermal amplification (LAMP): a versatile technique for detection of micro-organisms. Journal of Applied Microbiology. 2018; 124:626-643.

1. A CRISPR RNA (crRNA) applicable in a CRISPR-based ribonucleoprotein (RNP) system for reacting towards one or more genetic materials derived from Burkholderia pseudomallei in a biological sample comprising:
a spacer region having one of a polynucleotide sequence selected from SEQ ID No. 3, SEQ ID No. 5, and SEQ ID No. 7;
a repeat region preceding the spacer region forming a secondary structure thereof; and
a first extension region arranged immediately after the spacer region.

2. The crRNA of claim 1, wherein the spacer region is being configured to hybridize on a complementary sequence resided in the genetic materials and the complementary sequence is preceded with a protospacer adjacent motif (PAM) comprising a sequence of TTTV with V being a non-T nucleotide.

3. The crRNA of claim 1 further comprising a second extension region having a sequence of GGG being arranged preceding of the repeat region.

4. The crRNA of claim 1, wherein the repeat region comprises a polynucleotide sequence of SEQ ID No. 9 or SEQ ID No. 10.

5. The crRNA of claim 1, wherein the first extension region comprises a polynucleotide sequence of AAAGGAA.

6. The crRNA of claim 1, wherein the CRISPR-based RNP system is a CRISPR-Cas12a RNP system.

7. A method for detecting presence of one or more genetic materials derived from Burkholderia pseudomallei acquired from a biological sample comprising the steps of:
providing the one or more genetic materials derived from Burkholderia pseudomallei;
assembling of a CRISPR-based RNP system, the assembled CRISPR-based RNP system comprising a crRNA with a spacer region having one of polynucleotide sequences selected from SEQ ID No. 3, SEQ ID No. 5, and SEQ ID No. 7;
reacting the genetic materials with the assembled CRISPR-based RNP system along a plurality of reporter probes in a buffer solution, the CRISPR-based RNP system being configured to activate collateral cleavage the reporter probe for emitting a signal upon hybridizing on a polynucleotide sequence complementary to the spacer region located within the one or more genetic materials; and
detecting the emitted signal to confirm presence of the Burkholderia pseudomallei in the biological sample.

8. The method of claim 7, wherein the spacer region is being configured to hybridize on a complementary sequence resided in the genetic materials and the complementary sequence is preceded with a protospacer adjacent motif (PAM) comprising a sequence of TTTV with V being a non-T nucleotide.

9. The method of claim 7, wherein the detecting step further comprises subjecting portion of the reacted buffer solution to a lateral flow assay for capturing the emitted signal.

10. The method of claim 7, wherein the crRNA further comprises a repeat region preceding the spacer region forming a secondary structure thereof and the repeat region comprises a polynucleotide sequence of SEQ ID No. 9 or SEQ ID No. 10.

11. The method of claim 7, wherein the crRNA further comprises a first extension region arranged immediately after the spacer region and the first extension region comprises a polynucleotide sequence of AAAGGAA.

12. The method of claim 7, wherein the crRNA further comprises a second extension region having a sequence of GGG being arranged preceding of the repeat region.

13. The method of claim 7, wherein the CRISPR-based RNP system is a CRISPR-Cas12a RNP system.
